(19) United States
(12) Patent Application Publication
(10) Pub. No.: US 2012/0315670 A1
(43) Pub. Date: Dec. 13, 2012

(54) Compositions and Methods for the Regulation of Multiple Genes of Interest in a Cell

(75) Inventors: Joseph Jacobson, Newton, MA (US); George Church, Brookline, MA (US)

(73) Assignee: Gen9, Inc., Cambridge, MA (US)

(21) Appl. No.: 13/505,376

(22) PCT Filed: Nov. 2, 2010
(86) PCT No.: PCT/US10/55079

§ 371 Date: Jun. 19, 2012

Related U.S. Application Data
(60) Provisional application No. 61/290,141, filed on Dec. 24, 2009.
(60) Provisional application No. 61/280,367, filed on Nov. 2, 2009.

(51) Int. Cl. C12P 21/00; C12N 15/63

(52) U.S. Cl. 435/69.1; 435/440

(57) ABSTRACT

Methods and compositions are provided for manipulating the genome of host cell to produce at least one exogenous gene product. Also provided are methods and composition for producing a programmable cell comprising a plurality of exogenous genes, wherein each exogenous gene is under the control of a disrupted regulatory sequence and wherein the disrupted regulatory sequences are restored by in vivo recombination. Preferably, the gene of interest is under the control of a genetically altered promoter which sequence recombination effects the expression of the exogenous gene(s).

### RELATED APPLICATIONS

This application claims the benefit under 35 U.S.C. §119(e) from U.S. provisional application Ser. No. 61/280,367, filed Nov. 2, 2009, and from U.S. provisional application Ser. No. 61/290,141, filed Dec. 24, 2009, the entire contents of which are herein incorporated by reference.

### FIELD OF THE INVENTION

Aspects of the invention relate to methods and compositions for genetically modifying cells. Aspects of the invention relate to methods and compositions for the expression of a plurality of exogenous genes in a host cell. More particularly, aspects of the invention relate to the filed of cell based programmable metabolic pathways.

### BACKGROUND

Manipulation of nucleic acids and regulation of the expression of proteins is an important aspect of modern molecular biology and functional genomics. Accordingly, there is a need for engineering techniques for the manipulation of the genetic content of a cell and for the rapid and planned expression of genes of interest in a cell. Such techniques would permit the development of cells with improved properties that can be used for analytical, research, industrial or therapeutic purposes.

### SUMMARY OF THE INVENTION

Aspects of the invention relate to the regulation of a set of predetermined exogenous genes in a host cell. Certain aspects of the invention relate to genes encoding proteins having novel function or to the regulation of theses genes. In certain embodiments, genes have novel regulatory elements. Aspect of the invention relate to the design of gene, set of genes, library of unrelated genes or libraries of variant genes that can be selectively activated in a host cell. Accordingly, aspects of the invention enable the generation of host cells with potential diverse functions. Once a function (or protein) is selected, the genetic material encoding the selected function is created by rearrangement of nucleic acid sequences. In certain embodiments, the genetic material is about 10 kilobases in length, about 50 kilobases in length, about 100 kilobases in length, about 500 kilobase sin length or longer. In some embodiments, the rearranged genetic material is a genome, such as a bacterial genome. Preferably, the rearrangement of nucleic acid sequences restores the function of a promoter which controls the expression of at least predetermined gene of interest. In an exemplary embodiment, the rearrangement restores the integrity of the promoter sequence.

Aspects of the invention relate to methods for manipulating the genome of host cells to produce at least one exogenous gene product. In some embodiments, a host capable of performing site directed recombination is provided. For example, the host cell expresses a set of recombinase enzymes under the control of a constitutive or inducible promoter. In some embodiments, two or more genetic elements are provided and introduced into the host cell. In some embodiments, the first genetic element comprises a genetically altered promoter sequence and at least one or more first sequence homologous to a DNA sequence. The first genetic element is operably linked to a second genetic element comprising at least part of a coding sequence. Under the genetically altered promoter-coding sequence configuration, the gene product is not expressed. Genetic alteration includes a mutation, an insertion, a deletion, a substitution, a reversion, a tranversion, a double-strand break. Under appropriate conditions, promoting site directed recombination between the disrupted promoter and the target DNA sequence, the promoter sequence is repaired leading to a functional promoter-coding sequence configuration. In some embodiments, the nucleic acid sequences are synthetic nucleic acids.

Preferably, the promoter sequence comprises homologous recombination sites flanking the altered sequence and the promoter sequence can be recombined by homologous recombination. In some embodiments, host cells are grown under conditions promoting recombination enabling the production of a gene product. In some embodiment, the gene product is linked to a detectable signal and host cells comprising recombined nucleic acid sequences can be selected and isolated. In some embodiments, a plurality of unrelated genes may be operably linked to a promoter sequence or to a plurality of promoter sequences. In other embodiments, a library of gene variants is operably linked to a promoter or a plurality of promoter. The plurality of genes may comprise recombination sites therebetween promoting the recombination and rearrangement of the different gene sequences. In preferred embodiments, homologous sequences flank the 3′ and the 5′ ends of one or more genetic element. Homologous sequences can be the identical or can be different. The plurality of promoters may be a library of unrelated promoters or a library of promoter variants. In certain embodiments, the genetic elements are provided in a vector, cosmid, or BAC vector. In a preferred embodiment, the host cell is a bacterium, preferably E. Coli. In some embodiments, the host cell expresses rec E and recT genes. In a preferred embodiment, the host cell is E. coli and the recombination is implemented by lambda Red recombination system. In some embodiments, the recombination is implemented by Cre/lex recombination system.

In some embodiments, the gene product is a mRNA. In other embodiments, the gene product is a protein, for example an enzyme. In some embodiments, the genetic element comprises a cluster of genes which codes for metabolic enzymes from a metabolic pathway.

Aspects of the invention provide components, nucleic acid preparations and engineered host cells for the selective expression of one or more genes. In some embodiments, the invention provide an engineered host cell comprising a plurality of genetic elements wherein the plurality of genetic elements does not exist in the native host cell and wherein at least one genetic element function is restored by in vivo recombination. According to aspects of the invention, an engineered host cell comprises a plurality of exogenous genetic elements, the first genetic element comprising at least one first genetically altered promoter sequence with one or more first homologous sequences and a second genetic element which sequence corresponds in part to a coding sequence. Preferably, the genetically altered promoter sequence comprises within its sequence homologous recombination sites which will allow the repair of the promoter sequence by homologous recombination, and thereby the expression of the second genetic element. The second genetic element may be a library of gene variants, a library of genes or a cluster of genes encoding enzyme from a metabolic pathway.

Aspects of the invention relate to the expression of one or more nucleic acid sequence of interest in a host cell by introducing a set of genetic elements in a host cell, each genetic elements having at least one recombination site and wherein the set of genetic elements comprises at least one regulatory sequence and at least one coding nucleic acid sequence of interest, exposing the cell under conditions promoting recombination, rearranging the set of genetic elements by allowing recombination between recombination sites and isolating the host cell expressing the at least one nucleic acid sequence of interest at a desired expression level. The genetic elements may be on a same or on different nucleic acids and the nucleic acids may be on a plasmid or integrated to the host cell genome. Preferably, the regulatory sequences are a promoter sequence. Expression of the nucleic acid sequence of interest is modulated by rearranging the regulatory sequences and/or the genetic elements. In some embodiments, the regulatory sequences are disrupted and the expression of selected coding sequences is modulated by restoring the function of at least one regulatory sequence. Preferably, the function of the regulatory sequence is restored by homologous recombination.

Some aspects of the invention relate to the design and engineering of a metabolic pathway, by providing a plurality of genetic elements, the plurality of genetic elements comprising (i) a plurality of regulatory sequences, wherein the regulatory sequences have different strength and (ii) a plurality of coding sequences, wherein each coding sequences encodes for a protein catalyzing each step of the metabolic pathway and wherein the plurality of genetic elements comprises homologous recombination sites therebetween; and rearranging the genetic elements by homologous recombination thereby operably linking each coding sequence to a regulatory sequence having an optimal strength. In some embodiments, at least one regulatory sequence is disrupted and its function by homologous recombination thereby allowing the expression of the coding sequences operably linked to the restored regulatory sequences.

Aspects of the invention relate to methods of producing a programmable engineered cell capable of expressing at least one nucleic acid sequence. The method comprises providing a cell comprising at least one exogenous nucleic acid sequence wherein the at least one nucleic acid sequence is linked to a regulatory nucleic acid sequence; providing at least one predefined oligonucleotide sequence homologous to the at least part of the regulatory nucleic acid sequence; exposing the cell to conditions promoting oligonucleotide-directed recombination; and selecting a cell expressing the at least one nucleic acid sequence. In some embodiments, the at least one nucleic acid sequence is operably linked to the regulatory sequence, such as a promoter sequence. In some embodiments, the at least one regulatory sequence is disrupted. The nucleic acid sequence can be an operon or a library of genes. In some embodiments, the nucleic acid sequence is a library of genes and the at least one oligonucleotide is a library of predefined oligonucleotides sequences. In some embodiments, the at least part of the regulatory sequence is replaced by homologous recombination. Recombination between homologous sequences can occur in parallel or serially.

Aspects of the invention relate to a programmable cell comprising a plurality of exogenous genes, wherein each exogenous gene is under the control of a disrupted regulatory sequence and wherein the disrupted regulatory sequences are restored by in vivo recombination or lambda red recombination.

Other aspects of the invention relate to a kit comprising a programmable cell, wherein the programmable cell comprises a plurality of exogenous genes, wherein each exogenous gene is under the control of a disrupted target regulatory sequence; and a plurality of predefined oligonucleotide sequences, wherein a portion of the oligonucleotide sequence is identical to a subsequence of a non-disrupted regulatory sequences and a portion of the oligonucleotide sequence is homologous to the target regulatory sequence.

### BRIEF DESCRIPTION OF THE DRAWINGS

FIG. 1. illustrates a non-limiting schematic representation of a method of expressing a gene of interest in a host cell, wherein the gene of interest is operably linked to an altered promoter.

FIG. 2 illustrates a non-limiting schematic representation of the generation of a plurality of genetic configurations by rearrangement of promoter and coding modules (A, B, C).

FIG. 3 illustrates a non-limiting schematic representation of a library of biological parts.

FIG. 4 illustrates a non-limiting schematic representation of oligonucleotide programmable microprocessor cell and associated programming oligonucleotides.

### DETAILED DESCRIPTION

Aspects of the invention relate to the generation of cells having a predetermined set of heterologous genetic elements and to the regulation of the heterologous genetic elements. In some aspects, the invention relates to the design of genetic elements for recombination in a host cell. Accordingly, aspects of the invention relate to methods and compositions for assembling large nucleic acids constructs in a predetermined order to modify or replace a host cell genome.

Aspects of the invention relate to a multipurpose engineered biological cell, which contains a plurality of nucleic acid sequences (e.g. library of nucleic acid sequences) or biological parts which may be programmed to perform a specific function. Other identical copies of the cell may be programmed to perform other functions. One skilled in the art would appreciate that engineered cells which may function in a way analogous to a multipurpose microprocessor in electronics. For example, engineered genetic circuitry may function in a way analogous to semiconductor based logic systems. Semiconductor based logic systems such as silicon based integrated circuits comprise generally two separate classes of circuit: I] Special purpose circuits and II] General purpose circuits. Special purpose circuits such as Application Specific Integrated Circuits (ASICs) are those in which individual transistors are hard wired at the time of manufacture in order to create a dedicated special purpose circuit. Such circuits may typically be designed in a Computer Aided Design (CAD) environment from a library of parts (e.g. an IP core library) from which a large number of different types of circuits may be defined. Such circuits typically have a specific task which they carry out and are not reprogrammable or only have limited re-programmability after manufacture. Such circuits typically have high performance for their intended task but if a substantially different task is required then one is required to redesign and re-fabricate a new circuit, a task which can be both expensive and time consuming.

The second class of circuit are general purpose circuits such as microprocessors and field programmable gate arrays (FPGAs) which can be programmed or reprogrammed though software to perform a variety of tasks. In such a system the characteristics of such circuits are generally made known and software engineers can design software to control the same microprocessor or FPGA to perform many different tasks.

Recently there has been considerable interest in creating logic circuits using parts from molecular biology instead of silicon. These endeavors fall into a field called Synthetic Biology which builds upon existing disciplines from molecular biology including genetic engineering and metabolic pathway engineering but in addition harnesses other engineering modalities including especially those from electrical engineering and computer science. For example, a nucleic acid sequence may be built from parts or subparts such as oligonucleotides, a transcription unit (an open reading frame plus regulatory elements), assemblies of multiple genes, or smaller polynucleotide sequences an open reading frame or portion thereof, or a regulatory segment. In some embodiments, the desired nucleic acid sequence is decomposed into a plurality of building blocks. In some embodiments, the genetic building blocks, functional building blocks or biological parts are designed by Computer Aided Design (CAD) software. A large collection, currently on the order of 3000, of biological parts is maintained by the Registry of Standard Biological Parts (partsregistry.org). To date biological circuits analogous to special purpose circuits created in silicon such as toggle switches (Gardner, T S et al., Nature, Vol. 403, pp 339-342, 2000) and ring oscillators (Elowitz & Leibler, Nature, Vol. 403, pp 335-338, (2000) Nature 403, pp 335-338) have been created using the transcriptional regulatory elements which control metabolic pathways within cellular biology. Such biological circuits are analogous to silicon based Application Specific Integrated Circuits (ASICs) in which individual transistors are hard wired in order to create a dedicated special purpose circuit.

As is the case in electrical circuits, although application specific biological circuits can have a high degree of performance for the particular functionality they are fabricated to carry out, they suffer from the requirement of having to re-synthesize or re-assemble the circuit if a significantly new functionality is desired. Such re-synthesis or reassembly can be costly and time consuming.

As used herein, the term “genome” refers to the whole hereditary information of an organism that is encoded in the DNA (or RNA for certain viral species) including both coding and non-coding sequences. In various embodiments, the term may include the chromosomal DNA of an organism and/or DNA that is contained in an organelle such as, for example, the mitochondria or chloroplasts and/or extrachromosomal plasmid and/or artificial chromosome. As used herein, a “gene” refers to a nucleic acid fragment that expresses a specific protein, including regulatory sequences preceding (5′ non-coding sequences) and following (3′ non-coding sequences) the coding sequence. A “native gene” refers to a gene that is native to the host cell with its own regulatory sequences whereas an “exogenous gene” or “heterologous gene” refers to any gene that is not a native gene, comprising regulatory and/or coding sequences that are not native to the host cell. In some embodiments, an heterologous gene may comprise mutated sequences or part of regulatory and/or coding sequences. In some embodiments, the regulatory sequences may be heterologous or homologous to a gene of interest. An heterologous regulatory sequence does not function in nature to regulate the same gene(s) it is regulating in the transformed host cell. “Coding sequence” refers to a DNA sequence coding for a specific amino acid sequence. As used herein, “regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include promoters, translation leader sequences, RNA processing site, effector binding site and stem-loop structure. As described herein, a genetic element may be any coding or non-coding nucleic acid sequence. In some embodiments, a genetic element is a nucleic acid that codes for an amino acid, a peptide or a protein. Genetic elements may be operons, genes, gene fragments, promoters, exons, introns, etc. or any combination thereof. Genetic elements can be as short as one or a few codons or may be longer including functional components (e.g. encoding proteins) and/or regulatory components. In some embodiments, a genetic element consists of an entire open reading frame of a protein, or consists of the entire open reading frame and one or more (or all) regulatory sequences associated with that open reading frame. One skilled in the art will appreciate that the genetic elements can be viewed as modular genetic elements or genetic modules. For example, a genetic module can comprise a regulator sequence or a promoter or a coding sequence or any combination thereof. In some embodiments, the genetic element comprises at least two different genetic modules and at least two recombination sites. In eukaryotes, the genetic element can comprise at least three modules. For example, a genetic module can be a regulator sequence or a promoter, a coding sequence, and a polyadenlylation tail or any combination thereof. In addition to the promoter and the coding sequences, the nucleic acid sequence may comprises control modules including, but not limited to a leader, a signal sequence and a transcription terminator. The leader sequence is a non-translated region operably linked to the 5′ terminus of the coding nucleic acid sequence. The signal peptide sequence codes for an amino acid sequence linked to the amino terminus of the polypeptide which directs the polypeptide into the cell's secretion pathway.

Genetic elements or genetic modules may derive from the genome of natural organisms or from synthetic polynucleotides or from a combination thereof. In some embodiments, the genetic elements modules derive from different organisms. Genetic elements or modules useful for the methods described herein may be obtained from a variety of sources such as, for example, DNA libraries, BAC libraries, de novo chemical synthesis, or excision and modification of a genomic segment. The sequences obtained from such sources may then be modified using standard molecular biology and/or recombinant DNA technology to produce polynucleotide constructs having desired modifications for reintroduction into, or construction of, a large product nucleic acid, including a modified, partially synthetic or fully synthetic genome. Exemplary methods for modification of polynucleotide sequences obtained from a genome or library include, for example, site directed mutagenesis; PCR mutagenesis; inserting, deleting or swapping portions of a sequence using restriction enzymes optionally in combination with ligation; in vitro or in vivo homologous recombination; and site-specific recombination; or various combinations thereof. In other embodiments, the genetic sequences useful in accordance with the methods described herein may be synthetic polynucleotides. Synthetic polynucleotides may be produced using a variety of methods such as high throughput oligonucleotide assembly techniques known in the art. For example, oligonucleotides having complementary, overlapping sequences may be synthesized on an array and then eluted off. The oligonucleotides then are induced to self assemble based on hybridization of the complementary regions. In some embodiments, the methods involve one or more nucleic assembly reactions in order to synthesize the genetic elements of interest. The method may use in vitro and/or in vivo nucleic assembly procedures. Non-limiting examples of nucleic acid assembly procedures and library of nucleic acid assembly procedure are known in the art and can be found in, for example, U.S. patent applications 20060194214, 20070231805, 20070122817, 20070269870, 20080064610, 20080287320, the disclosures of which are incorporated by reference.

In some embodiments, genetic elements sequence share less than 99%, less than 95%, less than 90%, less than 80%, less than 70% identity with a native or natural nucleic acid sequence. Identity can each be determined by comparing a position in each sequence which may be aligned for purposes of comparison. When an equivalent position in the compared sequences is occupied by the same base or amino acid, then the molecules are identical at that position; when the equivalent site occupied by the same or a similar amino acid residue (e.g., similar in steric and/or electronic nature), then the molecules can be referred to as homologous (similar) at that position. Expression as a percentage of homology, similarity, or identity refers to a function of the number of identical or similar amino acids at positions shared by the compared sequences. Expression as a percentage of homology, similarity, or identity refers to a function of the number of identical or similar amino acids at positions shared by the compared sequences. Various alignment algorithms and/or programs may be used, including FASTA, BLAST, or ENTREZ. FASTA and BLAST are available as a part of the GCG sequence analysis package (University of Wisconsin, Madison, Wis.), and can be used with, e.g., default settings. ENTREZ is available through the National Center for Biotechnology Information, National Library of Medicine, National Institutes of Health, Bethesda, Md. In one embodiment, the percent identity of two sequences can be determined by the GCG program with a gap weight of 1, e.g., each amino acid gap is weighted as if it were a single amino acid or nucleotide mismatch between the two sequences. Other techniques for alignment are described in Methods in Enzymology, vol. 266: Computer Methods for Macromolecular Sequence Analysis (1996), ed. Doolittle, Academic Press, Inc., a division of Harcourt Brace & Co., San Diego, Calif., USA. Preferably, an alignment program that permits gaps in the sequence is utilized to align the sequences. The Smith-Waterman is one type of algorithm that permits gaps in sequence alignments. See Meth. Mol. Biol. 70: 173-187 (1997). Also, the GAP program using the Needleman and Wunsch alignment method can be utilized to align sequences. An alternative search strategy uses MPSRCH software, which runs on a MASPAR computer. MPSRCH uses a Smith-Waterman algorithm to score sequences on a massively parallel computer.

In yet other embodiments, genetic elements or modules useful in accordance with the methods described herein may be excised from the genome and then modified as described above. It should be appreciated that the nucleic acid sequence of interest or the gene of interest may derive from the genome of natural organisms. In some embodiments, genes of interest may be excised form the genome of a natural organism or form the host genome, for example E. Coli. It has been shown that it is possible to excise large genomic fragments by in vitro enzymatic excision and in vivo excision and amplification. For example the FLP/FRT site specific recombination system and the Cre/loxP site specific recombination systems have been efficiently used for excision large genomic fragments for the purpose of sequencing (see, Yoon et al., Genetic Analysis: Biomolecular Engineering, 1998, 14: 89-95). In some embodiments, excision and amplification techniques can be used to facilitate artificial genome or chromosome assembly. Genomic fragments may be excised form E. Coli chromosome and altered before being inserted into the host cell artificial genome or chromosome. In some embodiments, the excised genomic fragments can be assembled with engineered promoters and inserted into the genome of the host cell.

In one aspect of the invention, methods are provided to alter a cell function or to generate a novel cell function by introducing nucleic acid sequences comprising a set of genetic elements or genetic modules having recombination sites situated therebetween, rearranging the genetic elements by recombination at the recombination sites and selecting the cells in which the recombination has occurred. In some embodiments, the recombinant cells express one or more polypeptide of interest (e.g. library of polypeptides of interest). Expression will be understood to include any step involved in the production of the polypeptide including, but not limited to, transcription, post-transcriptional modification, translation, post-translational modification, and secretion. In preferred embodiments, the genetic elements comprise one or more recombination sites. In preferred embodiments, genetic elements are introduced into a host cell genome by site directed recombination. The genetic element may comprise a plurality of coding sequences of interest and/or a least one engineered regulatory sequence. In some embodiments, the plurality of genetic elements comprises one or more genes of interest linked together with recombination sites. In some embodiments, the plurality of genes is a library of unrelated genes. For example, the unrelated genes may be genes of a metabolic pathway. In other embodiments, the plurality of genes is a library of gene variants. In some exemplary embodiments, the plurality of genes of interest is operably linked to one promoter or each of the plurality of gene is operably linked to a different promoter. In some embodiments, genetic elements and/or genetic modules are flanked with recombination sites. For example, the recombination sites are flanking the genetic element or genetic modules at its 5′ and 3′ end. Yet in preferred embodiments, the genetic elements comprise a sequence that may be used as a recombination site (e.g, the recombination sites are part of the genetic element sequence). Each genetic element or genetic module can be flanked with one or more unique recombination sites thereby allowing each genetic module to be assembled in a predetermined order. For example, a cluster of genetic modules corresponding to a library of genes may be assembled and be placed under the control of a unique selected promoter. It should be appreciate that genetic elements can comprise a cluster of unrelated genes or a cluster of gene variants. In some embodiments, genetic elements may comprise a plurality of genes that are organized in one or more operons. As used herein the term “operon” refers to a nucleic acid sequence comprising several genes that are clustered and optionally transcribed together into a polycistronic mRNA, e.g. gene encoding for the enzymes of a metabolic pathway. In some embodiments, genetic elements may comprise a cluster of different promoters or of promoter variants. Recombination sites may be located within the genetic elements, immediately adjacent to the genetic element or may be linked to the genetic element via a linker sequence. The linker sequence may be of a bout 10, about 50, about 100 nucleotides in length. Recombination sites may be at lest 6, at least 8, at least 10, at least 20, at least 30, at least 40, at least 50, at least 100 base pairs long.

The genetic elements are preferably heterologous genomic sequences that may reside in the host cell as extrachromosomal or intrachromosomal nucleic acid sequences. In some embodiments, the genome of the host cell is modified by insertion or replacement of the part of the native genome with genetic elements through recombination. In some embodiments the artificial genome is a minimal genome. In some embodiment, the artificial chromosome or artificial genome comprises at least one genetic element of interest which sequence is not naturally found in the host cell. In a preferred embodiment, the artificial chromosome comprises a plurality of genetic elements of interest which sequences are not naturally found in the host cell. In preferred embodiments, genetic elements of interest are grouped together with one or more genetic regions (for example, plasmid, phage, vector, chromosome, genomic region). In some embodiments, artificial genomes or chromosomes size ranges from 1 to 10 kb, 5 to 50 kb, 50 to 100 kb, 100 to 800 kb or 1 Mbp or larger. According to some aspects of the method, the genetic modules are designed, synthesized and assembled to form at least part of the artificial genome or artificial chromosome. For example, at least 2, at least 5, at least 10, at least 20, at least 50, at least 100 genetic elements may be assembled. Preferably, the components are assembled by in vivo recombination. When assembled, theses sequences may be referred as artificial genomic sequences, artificial chromosome or artificial genome. As used herein, artificial genome, artificial chromosomes and modified genome are used interchangeably and refer to large genomic sequences that are not found naturally in the host genome. In some aspects of the invention, part or the whole natural genome of the parental host cell is removed and replaced by the assembled engineered genome. Further aspects of the invention relate to modified host cells hosting genetic elements or libraries of genetic elements of the invention and allowing for recombination of the genetic elements.

Components of the artificial genome or chromosome may be assembled in a wide variety of organism or host cells. The host organism may be a prokaryotic organism. Examples of prokaryotic organism include, but are not limited to, Escherichia, Bacillus, Pseudomonas, Lactococcus, Streptococcus, Enterococcus, and Lactobacillus. In particular interesting strains include, but are not limited to, Escherichia coli, Bacillus subtilis, Mycobacterium Jannaschii., Corynebacterium glutanicum Preferably, the host organism is a bacterium, for example E. Coli (e.g. E. Coli strain K-12). Yet in other embodiment, example of host cells include, but is not limited to, insect cells such as Drosophila melanogaster cells, plant cells, yeast cells (e.g. Saccharomyces cerevisiae, Sacharomyyces pombe, Pichia species, Candida species), Archae, amphibian cells such as Xenopus laevis cells, nematode cells such as Caenorhabditis elegans cells, or mammalian cells (such as Chinese hamster ovary cells (CHO), mouse cells, African green monkey kidney cells (COS), fetal human cells (293T) or other human cells). Other suitable host cells are known to those skilled in the art.

Aspects of the invention relate to the regulation of at least one heterologous gene of interest in a host cell. In preferred embodiments, the coding nucleic acids sequences are under the control of one or more regulatory elements. Regulatory regions include for example promoters, replication of origins, terminators, and/or repressors. In some embodiments, a regulatory component is inducible and responds to an internal or an external signal. It is well known that regulatory elements may exert a negative or positive control on the expression of a nucleic acid sequence. In an exemplary embodiment, expression of a gene of interest may be altered by altering the regulatory regions that are operably linked to the gene of interest. For example, a negative or positive control may be exerted indirectly by decreasing or increasing transcription, mRNA stability, and/or translation of the nucleic acid sequence. The alteration of the regulatory regions may be achieved by changing the promoter strength or by using regulatable (e.g inducible, activatible) promoters that may be induced following the treatment of a host cell with an agent, biological molecule, chemical, ligand, light, temperature or the like. As used herein, an “inducer” refers to an agent that initiates transcription or increases the rate of transcription of a gene of interest. One should appreciate that in some applications, such as metabolic engineering, fine tuning the expression of specific genes is critical to control the metabolic flux. It may be therefore be useful to design and use different promoters with variable strength or with a slightly lower or higher strength than wild type. In some embodiments, the regulatory sequence may be a combination of constitutive and regulatable promoters. Yet in other embodiments, the regulatory sequence comprises one, two, three or more promoter sequences (e.g. tandem promoter sequences). In a preferred embodiment, regulatory elements are used to finely tune the expression of gene(s) of interest. A number of methods have been developed to allow the modulation of genes in E. Coli. For example, libraries of promoters having different strength have been developed for bacterial host cells (see for example, U.S. Pat. No. 7,199,233 and US 20060014146). In some embodiments, the promoter strength may be tuned to be appropriately responsive to activation or inactivation. Yet in other embodiments, the promoter strength is tuned to constitutively allow an optimal level of expression of a gene of interest or of a plurality of gene of interest.

Aspects of the invention relate to the design and assembly of genetic elements comprising a disrupted or altered promoter as well as to the manipulation and use of disrupted or altered promoters to control gene(s) expression in a host cell. Further aspects of the invention relate to the organism or host cells which contain such constructs. Accordingly, aspects of the invention relate to promoter activation, suppression and/or fine tuning. In some embodiments, the promoter may be a mutated, a truncated, a hybrid, or a disrupted promoter. In some aspects of the invention, the engineered cell is capable of synthesizing genes products of interest under conditions that restore promoter functionality as described herein. Some aspects of the invention provide methods for rationally designing promoter sequences that are functional after sequence modification. As used herein “promoter” refers to a DNA sequence capable of controlling the level of expression of a coding sequence or functional RNA. For transcription to take place, an RNA polymerase attached to a promoter sequence. The promoter sequence provides a binding site for the RNA polymerase and for transcription factors. In general, a coding sequence is located 3′ to a promoter sequence. Promoters may be derived in their entirety from a native gene, or be composed of different elements derived from different promoters found in nature, or even comprise synthetic nucleic acid sequences. Any promoter element may be used to drive the expression of a specific gene. Promoters include prokaryotic promoters and eukaryotic promoters. Suitable prokaryotic promoters include but are not limited to promoter form the E. Coli lac operon, promoter of the Bacillus lentus alkaline protease gene (aprH), promoter of the Bacillus subtilis alpha-amylase gene, promoter of the beta-lactamase gene, tac promoter, etc. The promoter can be a constitutive promoter, an inducible promoter or a cell type specific promoter. In some embodiments, promoters are inducible, for example Ptrc which is induced by IPTG. Ina preferred embodiment, inducible promoters are used when the gene product is toxic to the host cell. One should appreciate that promoters have modular architecture and that the modular architecture may be altered. Bacterial promoters typically include a core promoter element and additional promoter elements. The core promoter refers to the minimal portion of the promoter required to initiate transcription. A core promoter includes a Transcription Start Site, a binding site for RNA polymerases and general transcription factor binding sites. The “transcription start site” refers to the first nucleotide to be transcribed and is designated +1. Nucleotides downstream the start site are numbered +1, +2, etc., and nucleotides upstream the start site are numbered −1, −2, etc. Additional promoter elements are located 5′ (i.e. typically 30-250 bp upstream the start site) of the core promoter and regulate the frequency of the transcription. The proximal promoter elements and the distal promoter elements comprises specific transcription factor site. In prokaryotes, a core promoter usually includes two consensus sequences, a −10 sequence or a −35 sequence, which are recognized by sigma factors (see, for example, Hawley; D. K. et al (1983) Nucl. Acids Res. 11, 2237-2255). The −10 sequence (10 bp upstream from the first transcribed nucleotide) is typically about 6 nucleotides in length and is typically made up of the nucleotides adenosine and thymidine (also known as the Pribnow box). In some embodiments, the nucleotide sequence of the −10 sequence is 5′-TATAAT or may comprise 3 to 6 bases pairs of the consensus sequence. The presence of this box is essential to the start of the transcription. The −35 sequence of a core promoter is typically about 6 nucleotides in length. The nucleotide sequence of the −35 sequence is typically made up of the each of the four nucleosides. The presence of this sequence allows a very high transcription rate. In some embodiments, the nucleotide sequence of the −35 sequence is 5′-TTGACA or may comprise 3 to 6 bases pairs of the consensus sequence. In some embodiments, the −10 and the −35 sequences are spaced by about 17 nucleotides. Eukaryotic promoters are more diverse than prokaryotic promoters and may be located several kilobases upstream of the transcription starting site. Some eukaryotic promoters contain a TATA box (e.g. containing the consensus sequence TATAAA or part thereof), which is located typically within 40 to 120 bases of the transcriptional start site. One or more upstream activation sequences (UAS), which are recognized by specific binding proteins can act as activators of the transcription. Theses UAS sequences are typically found upstream of the transcription initiation site. The distance between the UAS sequences and the TATA box is highly variable and may be up to 1 kb.

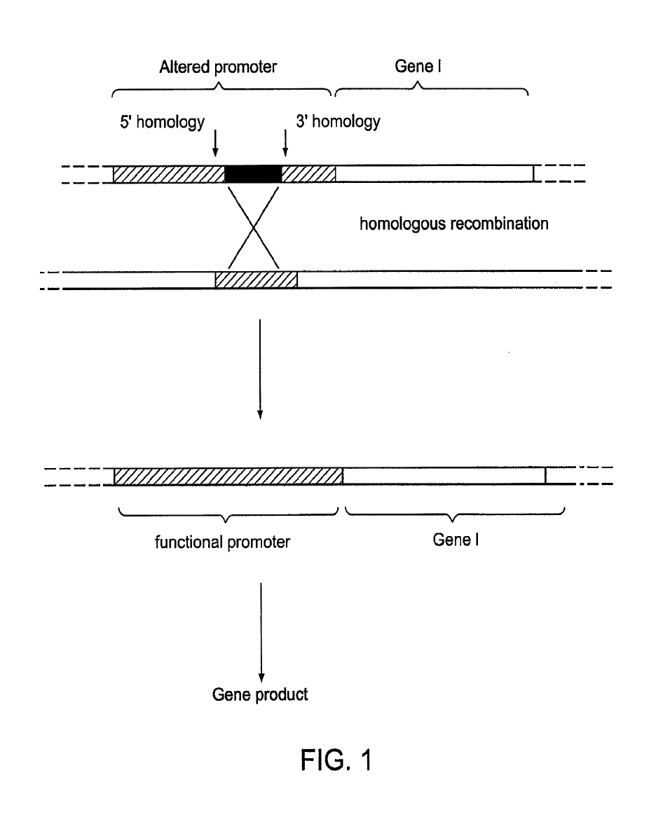

As used herein, the term “disruption” refers to any procedure to add, remove, substitute or alter genetic material in a genetic element, thereby influencing the expression of gene(s). In a preferred embodiments, host cells harboring a disrupted genetic element produces preferably at least less than 50%, less than 75%, less than 85%, less than 90%, less than 95% of the gene product compared host cells harboring non-disrupted genetic elements. Disruption and alteration are used herein interchangeably. As used herein an “nucleic acid sequence alteration” refers to any change in a nucleic acid sequence or structure, including but not limited to a deletion, an addition, a substitution, an insertion, a reversion, a transversion, a point mutation, a methylation. The disruption of the genetic elements can be achieved in any number of ways apparent to one skilled in the art, including, but not limited to targeted mutagenesis, site specific recombination and gene trapping. The portion of the genetic element to be altered may be, for example, the coding sequence and/or a regulatory element upstream or downstream of the coding sequences (e.g. promoter, transcription terminator, polyadenylation sequences, etc.). One or more nucleotides may be inserted or removed resulting in the introduction of a stop codon, the removal of a start codon, or a frame-shift of the open reading frame. For example, Datensko et al. have developed a method for disrupting chromosomal genes in E. coli in which PCR primers provide homology to the targeted gene(s) (PNAS, 2000, 97: 6640-6645). In a preferred embodiment, the promoter sequence is altered or disrupted. One would appreciate that disruption of the promoter is likely to be correlated with modified (e.g. decrease) promoter function and modified gene expression. In preferred embodiments, selected genes of interest are placed downstream of promoters that are engineered or disrupted to be non-functional, thereby inhibiting selected gene transcription. In a preferred embodiment, the selected gene(s) expression is totally inhibited before modification or repair of genes regulatory elements. In some embodiments, the promoter is engineered such as its activity is decreased by at least 90%, at least 95%, at least 98%, at least 99%. In some aspect of the invention, the promoter is engineered to be disrupted by mutations, substitutions, insertions or deletions (e.g. gap). For example, at least 1, 2, 5, 10, 20, 50, 100 nucleotides may be substituted, deleted, inserted, inverted within the promoter sequence. In an illustrative embodiment, the promoter sequences are disrupted by site specific recombination systems, thereby inactivating the expression of the genes that are operably linked to the disrupted promoters. For example, part of the promoter sequence may be inverted, or the promoter sequences may be interrupted by a double-strand break. In some embodiments, one or more consensus sequences are altered. For example, the −10 and/or the −30 sequences may be altered in prokaryotic promoters. In some embodiments, the recognition binding site for transcription factors and/or the activator biding site may be altered in the eukaryotic promoters. In other embodiments, the space region between the consensus regions is altered. In other embodiments, the hinge region between the recognition and the activator binding sites is altered. For example, the hinge region may be render more flexible or less flexible than the native hinge region. It has been shown that the spacer sequences surrounding the consensus −10 and −30 regions of promoters contribute to the promoter strength (Jensen et al., 1998, Appl. Env. Microbiol. 64-82-87). For example, the optimal distance between the −35 and the −10 hexamers of the promoters recognized by the RNA polymerase Sigma is usually 17 bp. Accordingly, the spacer region between the two hexamers regions may be increased or decreased. For example, the distance between the −30 and the −10 regions may comprise at least 18 bp, at least 19 bp, at least 20 bp, at least 30 bp, at least 40 bp, at least 50 bp. In another example, the distance between the two hexamer regions is less than 17 bp, less than 16 bp, less than 15 bp, less than 14 bp, less than 13 bp, less than 12 bp, less than 10 bp. In some other embodiments, the prokaryotic consensus −10 and/or the −30 sequences are mutated. Mutations may include deletions, insertions or substitutions. In some embodiments, the mutation allows a mutated nucleotide in the core promoter sequence to look more like the consensus sequence. Mutation of this kind generally makes the promoter stronger allowing the RNA polymerase to form a tighter bind to the DNA and thereby up-regulating the transcription. In other embodiments, mutations destroy conserved nucleotides in the consensus sequence. For example, the consensus sequence may be randomized. This kind of mutation generally makes the RNA polymerase bind in a less tightly fashion and thereby resulting in the down-regulation of the transcription. In some embodiments, the engineered promoter comprises the minimal promoter sequences, which are necessary to the promoter function, but not the regulatory elements. In preferred embodiments, the minimal promoter function is altered. In an exemplary embodiment, the alteration corresponds to a deletion. In other embodiments, promoter element sequences may be inverted or permuted. For example, the regulatory sequences may be placed downstream to the consensus sequences. Yet, in other embodiments, an additional sequence can be placed between the consensus sequences or between the consensus sequences and the transcription sites. In an exemplary embodiment, the additional sequence comprises a detectable marker thereby allowing identification of the cells comprising the non-functional promoter. In some embodiments, repair of the non-functional promoter sequence leads to the destruction of the selectable or detectable marker. In this case, the presence of a functional promoter may be determined by assaying the absence of the detectable or selectable marker. One should appreciate that the altered promoter function can be restored by desired rearrangement of the promoter sub-sequences (e.g. two sub-sequences are brought together by recombination). For example, the promoter function can be restored by integration of an additional sequence (e.g. the desired sequence is inserted at the position of the deleted sequence, see for example FIG. 1), by addition of an sequence (e.g. the additional sequence restoring the functionality of a truncated promoter), by excision (e.g. the additional sequence altering the promoter function is removed) or by inversion or permutation (e.g. the order of the consensus sequences and/or the regulatory sequences is restored). In some embodiments, a disruptive nucleic acid sequence together with recombination sites may be inserted into the promoter sequence. In some embodiments, selected promoters function is restored by in vivo recombination and operably linked genes are activated. In some embodiments, the promoter comprises recombination sequences flanking the altered promoter sequence. Promoter elements can then be rearranged by allowing recombination between the recombination sites (e.g. intramolecular recombination) and thereby restoring the promoter functionality. In some embodiments, promoter sequence is interrupted by a double strand break or double strand gap. In some embodiments, the double stranded break comprises a recognition site for a meganuclease and double strand break can be repaired by site directed recombination such as in vivo recombination thereby generating an intact and functional promoter sequence. In some embodiments, oligonucleotides comprising 5′ and 3′ ends homologous to the sequences flanking the double strand break direct the promoter repair.

In some embodiments, a library of promoter sequences is provided. In some embodiments, the library of promoters comprises a plurality of different promoters. Different promoters' sequences may be related or unrelated. In an exemplary embodiment, the promoter sequences may be obtained from a bacterial source. Each promoter sequence may be native or foreign to the polynucleotide sequence which it is operably linked to. Each promoter sequence may be any nucleic acid sequence which shows transcriptional activity in the host cell. A variety of promoters can be utilized. For example, the different promoter sequences may have different promoter strength. In some embodiments, the library of promoter sequences comprises promoter variant sequences. In a preferred embodiment, the promoter variants cover a wide range of promoter activities form the weak promoter to the strong promoter. A promoter used to obtain a library of promoters may be determined by sequencing a particular host cell genome. Putative promoter sequences may be then be identified using computerized algorithms such as the Neural Network of Promoter Prediction software (Demeler et al. (Nucl. Acids. Res. 1991, 19:1593-1599). Putative promoters may also be identified by examination of family of genomes and homology analysis. The library of promoter may be placed upstream of a single gene or operon or upstream of a library of genes. Preferably, the library of promoter comprises recombination sites. For example, the library of promoter sequences is flanked by recombination sites. In some embodiments, recombination sites may be present within the promoter sequences. Any combination of any appropriate number of genetic elements (e.g. promoters) and recombination sites may be used. In some embodiments, the recombination sites are the same. Yet in other embodiment, the recombination sites are different. Preferably, the recombination sites that are present within the promoter sequence are different from the recombination sites that are flanking the promoter sequences. In a preferred embodiment, the library of promoters operably linked to the gene(s) of interest is integrated in the host cell genome. Flanking homologous recombination sites replace the homologous regions at the target site in a host chromosome or plasmid. Preferably, the integration is stable.

In some embodiments, the engineered genetic elements are cloned into cloning vectors. For example, the polynucleotide constructs may be introduced into an expression vector and transfected into a host cell. Any suitable vector may be used. Appropriate cloning vectors include, but are not limited to, plasmids, phages, cosmids, bacterial vector, bacterial artificial chromosomes (BACs), P1 derived artificial chromosomes (PACs), YAC, P1 vectors and the like. Standard recombinant DNA and molecular cloning techniques used here are well known in the art and are described by Sambrook, J., Fritsch, E. F. and Maniatis, T., Molecular Cloning: A Laboratory Manual, Second Edition, Cold Spring Harbor Laboratory Press, Cold Spring Harbor, N.Y. (1989) (hereinafter “Maniatis”); and by Silhavy, T. J., Bennan, M. L. and Enquist, L. W., Experiments with Gene Fusions, Cold Spring Harbor Laboratory Cold Press Spring Harbor, N.Y. (1984); and by Ausubel, F. M. et al., Current Protocols in Molecular Biology, published by Greene Publishing Assoc. and Wiley-Interscience (1987). In some embodiments, a vector may be a vector that replicates in only one type of organism (e.g., bacterial, yeast, insect, mammalian, etc.) or in only one species of organism. Some vectors may have a broad host range. Some vectors may have different functional sequences (e.g., origins or replication, selectable markers, etc.) that are functional in different organisms. These may be used to shuttle the vector (and any nucleic acid fragment(s) that are cloned into the vector) between two different types of organism (e.g., between bacteria and mammals, yeast and mammals, etc.). In some embodiments, the type of vector that is used may be determined by the type of host cell that is chosen. Preferably, bacterium is used as a host cell and BAC vectors are utilized because of their capability to contain long nucleic acid sequences insert, typically, 50 to 350 kb (see Zhao et al., editors, Bacterial Artificial Chromosomes, Humana Press. Totowa, N.J. 2004, which is incorporated herein by reference).

In some embodiments, nucleic acid fragments may be assembled using site specific or in vivo recombination systems. Examples of site-specific recombination include, but are not limited to: 1) chromosomal rearrangements that occur in Salmonella typhimurium during phase variation, inversion of the FLP sequence during the replication of the yeast 2 μm circle, and in the rearrangement of immunoglobulin and T cell receptor genes in vertebrates, 2) integration of bacteriophages into the chromosome of prokaryotic host cells to form a lysogen, and 3) transposition of mobile genetic elements (e.g., transposons) in both prokaryotes and eukaryotes. Recombination systems use recombinase enzymes that catalyze the recombination. For example, RecA and the RecBCD pathways are used in bacteria to repair of DNA double strand breaks. RAD51 and DMC1 catalyses the repair of DNA double strand breaks in Eukaryotic cells. Different recombination systems may be useful to practice aspects of the invention. A site-specific recombinase is an enzyme that recognizes short DNA sequences that become the crossover regions during the recombination event and include recombinases, transposases, and integrases. In some embodiments, linear genetic elements are used. Yet in other embodiments, circular genetic elements are used. In some embodiments, genetic elements comprise an origin of replication. Genetic elements may be the flanked by regions homologous to regions of the host genome or to regions homologous to a target DNA. In some embodiments, the genetic elements are designed to comprise at each end a 20-50 bp nucleic acid sequence homologous to a target site in a nucleic acid sequence (e.g. vector sequence, genomic sequence, or other nucleic acid sequence). The target site refers to the predetermined genomic location where integration of the genetic element is to occur. In other embodiments, the target sequence is designed to comprise recombination sites that are homologous to the genetic element to be inserted. In some embodiments, multiple copies of the recombination site are inserted to increases the likelihood of a recombination event. In other embodiments, genetic elements are flanked by at least two different recombination sites. Having different recombination sites has the advantage that more than one recombination event can be triggered independently. Any combination of recombination sites (e.g., restriction sites, homologous sequences, etc.) can be used when assembling these different recombination sites.

Aspects of the invention use in vivo recombination systems to insert engineered components into the host cell genome or into an artificial genome. In some embodiments, linear components are recombined in E. coli using the lambda red recombination system (see U.S. Pat. Nos. 6,509,156, 6,355,412, 7,144,734 which are incorporated herein in their entirety). In other embodiments, linear recombination system using phage integrase may be used. Phage integrases catalyze the unidirectional site-specific recombination between two DNA recognition sequences, the phage attachment site, attP, and the bacterial attachment site, attB. Commercial recombination systems using att-integrase includes the Gateway system (InVitrogen).

In certain embodiments, a recombination site is a sequence-specific recombination site (e.g., a lox P site) that is recognized by a recombinase (e.g., the Cre enzyme). In general, the Cre-Lox recombination system is a type of site-specific recombination that involves first inserting a loxP site that contains specific binding sites for Cre recombinase into a genome and then splicing in a nucleic acid sequence of interest. It should be appreciated that the Cre-Lox system can be used as a genetic tool to control site specific recombination events in genomic nucleic acid, delete undesired nucleic acid sequences, and modify chromosome architecture.

The Cre/loxP recombination-mediated cassette exchange recombination system may be used with circular genetic elements. The Cre protein catalyzes recombination of DNA between two loxP sites and is involved in the resolution of P1 dimers generated by replication of circular lysogens (Sternberg et al. (1981) Cold Spring Harbor Symp. Quant. Biol. 45: 297). Cre can function in vitro and in vivo in many organisms including, but not limited to, bacteria, fungi, and mammals (Abremski et al. (1983) Cell 32: 1301; Sauer (1987) Mol. Cell. Biol. 7: 2087; and Orban et al. (1992) Proc. Natl. Acad. Sci. 89: 6861). The loxP sites may be present on the same DNA molecule or may be present on different DNA molecules; the DNA molecules may be linear or circular or a combination of both. The loxP site consists of a double-stranded 34 bp sequence which comprises two 13 bp inverted repeat sequences separated by an 8 bp spacer region (Hoess et al. (1982) Proc. Natl. Acad. Sci. USA 79: 3398 and U.S. Pat. No. 4,959,317). The internal spacer sequence of the loxP site is asymmetrical and thus, two loxP sites can exhibit directionality relative to one another (Hoess et al. (1984) Proc. Natl. Acad. Sci. USA 81: 1026). When two loxP sites on the same DNA molecule are in a directly repeated orientation, Cre excises the DNA between these two sites leaving a single loxP site on the DNA molecule (Abremski et al. (1983) Cell 32: 1301). If two loxP sites are in opposite orientation on a single DNA molecule, Cre inverts the DNA sequence between these two sites rather than removing the sequence. Two circular DNA molecules each containing a single loxP site will recombine with one another to form a mixture of monomer, dimer, trimer, etc. circles. The concentration of the DNA circles in the reaction can be used to favor the formation of monomer (lower concentration) or multimeric circles (higher concentration). The Cre protein has been purified to homogeneity (Abremski et al. (1984) J. MoI. Biol. 259: 1509) and the cre gene has been cloned and expressed in a variety of host cells (Abremski et al. (1983), supra). Purified Cre protein is available from a number of suppliers (e.g., Novagen and New England Nuclear/DuPont). The Cre protein also recognizes a number of variant or mutant lox sites (variant relative to the loxP sequence), including the loxB, loxL and loxR sites which are found in the E. coli chromosome (Hoess et al. (1982), supra). Other variant lox sites include loxP511 (Hoess et al. (1986), Nucleic Acids Res. 14: 2287-300), loxC2 (U.S. Pat. No. 4,959,317), loxΔ86, loxΔl 17, loxP2, loxP3, loxP23, loxS, and loxH. Cre catalyzes the cleavage of the lox site within the spacer region and creates a six base-pair staggered cut (Hoess and Abremski (1985) J. MoI. Biol. 181: 351). The two 13 bp inverted repeat domains of the lox site represent binding sites for the Cre protein. If two lox sites differ in their spacer regions in such a manner that the overhanging ends of the cleaved DNA cannot reanneal with one another, Cre cannot efficiently catalyze a recombination event using the two different lox sites. For example, it has been reported that Cre cannot recombine (at least not efficiently) a loxP site and a loxP511 site; these two lox sites differ in the spacer region. Two lox sites which differ due to variations in the binding sites (i.e., the 13 bp inverted repeats) may be recombined by Cre provided that Cre can bind to each of the variant binding sites. The efficiency of the reaction between two different lox sites (varying in the binding sites) may be less efficient than that between two lox sites having the same sequence (the efficiency, will depend on the degree and the location of the variations in the binding sites). For example, the loxC2 site can be efficiently recombined with the loxP site, as these two lox sites differ by a single nucleotide in the left binding site.

In other embodiments, the recombination system FLP-FRT is used which uses recombination sequences between short Flippase Recognition Target (FRT) sites by the Flippase recombination enzyme (FLP or Flp) derived from the 2μ, plasmid of the baker's yeast Saccharomyces cerevisiae (see Zhu X D, Sadowski P D (1995). “Cleavage-dependent Ligation by the FLP Recombinase”. J Biol Chem 270: 23044-23054). Like the loxP site, the frt site comprises two 13 bp inverted repeats separated by an 8 bp spacer. The FLP gene has been cloned and expressed in E. coli (Cox, supra) and in mammalian cells (PCT Publication No.: WO 92/15694) and has been purified (Meyer-Lean et al. (1987) Nucleic Acids Res. 15: 6469; Babineau et al. (1985) J. Biol. Chem. 260: 12313; and Gronostajski and Sadowski (1985) J. Biol. Chem. 260: 12328); the Int recombinase of bacteriophage lambda (with or without Xis) which recognizes att sites (Weisberg, et al., “Site-specific recombination in Phage Lambda,” In: Lambda II, Hendrix, et al. Eds., Cold Spring Harbor Press, Cold Spring Harbor, N.Y. (1983) pp. 211-250); the xerC and xerD recombinases of E. coli which together form a recombinase that recognizes the 28 bp dif site (Leslie and Sherratt (1995) EMBO J. 14: 1561); the Int protein from the conjugative transposon Tn916 (Lu and Churchward (1994) EMBO J. 13: 1541); Tpnl and the β-lactamase transposons (Levesque (1990) J. Bacteriol. 172: 3745); the Tn3 resolvase (Flanagan et al. (1989) J. MoI. Biol. 206: 295 and Stark et al. (1989) Cell 58: 779); the SpoIVC recombinase of Bacillus subtilis (Sato et al. (1990) J. Bacteriol. 172: 1092); the Hin recombinase (Galsgow et al. (1989) J. Biol. Chem. 264: 10072); the Cin recombinase (Hafter et al. (1988) EMBO J. 7: 3991); and the immunoglobulin recombinases (Malynn et al. Cell (1988) 54: 453).

In some embodiments, a plurality of genes having recombination sites is inserted into an artificial chromosome or a genomic region of a host cell. In some embodiments, the recombination sites are identical. In other embodiments, the recombination sites comprise at least two different types of recombination sites. In some embodiments, the recombination sites are homologous recombination sites. In some embodiments, a recombination site is a restriction enzyme site (i.e., a site recognized by and/or cleaved by a restriction enzyme). After cleavage by a restriction enzyme, a restriction site can promote recombination. Restriction sites may be of any length (e.g., 4-20 base pairs). The longer the restriction site, the less frequently it will normally occur in a genome. Enzymes that cut these longer sequences are sometimes referred to as “rare cutters”. Suitable restriction enzyme sites may be found, for example, in a commercial catalog (e.g., New England Biolabs). Therefore in some embodiments, the recombination sites are long restriction sites. In some embodiments, the recombination sites correspond to I-Scel, I-Ceul, PI-PspI, PI-SceI, and/or NotI restriction sites or any suitable chimeric restriction enzyme restriction sites. Exemplary meganucleases/meganuclease cleavage sites that may be used in association with the hierarchical assembly methods and genome excision methods described herein include, for example, I-Scel (cut site: TAGGG_ATAAACAGGGTAAT), I-Dmol (cut site: GCCTTGCCGG_GTAAAGTTCCGGCGCG), I-Crel (cut site: CAAAACGTC_GT GAAGACAGTTTGGT), and I-DreI-3 (cut site: CAAAACGTC_GTAAAGTTCCGGCG CG) (see e.g., Chevalier B S, et al., MoI Cell. 2002 10: 895-905 (2002)). Most restriction enzymes induce a double strand break. However, the action of certain restriction enzymes results in a single strand nick only. A single strand nick also may promote recombination because the processing of this nick by a replication fork or DNA repair enzymes can induce a recombination event. It should be appreciated that for a restriction site to act as a recombination site in vivo, the appropriate restriction enzyme must also be present in the cell. The enzyme may be endogenous to the cell or may be ectopically expressed or introduced into the cell directly as a protein. Examples of recombination enzymes include but are not limited to tyrosine recombinases, serine recombinases, FIp, RecA, Pre (plasmid recombination enzyme) and ERCCl.

Recombination is promoted by homology between the recombination sites. Therefore, the greater the homology (e.g., either in length or percentage), the higher recombination frequency will be. In a preferred embodiment, the recombination sites share 100% identity (i.e., their nucleotide sequences are identical) or at least 50%, 60%, 70%, 80%, 85%, 90%, 95%, 96%, 97%, 98% or 99% homology. The nucleotide recombination site sequences may used to determine their propensity to participate in desired recombination events. For example, a particular recombination site can be designed to recombine specifically with only one other recombination site by selecting sequences that are rare and highly homologous (e.g. at least 95% homologous).

In some embodiments, recombinase genes may be carried by the natural host genome, by a plasmid or may be integrated in the artificial chromosome or genome. The term “recombinase” as used herein refers to an enzyme or a plurality of enzymes, active fragments or an active variants thereof, capable of identifying recognition sites within recombination sites and thereby capable of catalyzing recombination events. The terms “sequence-specific recombinase” and “site-specific recombinase” refer to enzymes that recognize and bind to a specific recombination site or sequence and catalyze the recombination of nucleic acid in relation to these sites. The “site-specific recombinase target site” refers to short nucleic acid site or sequence which is recognized by a sequence- or site-specific recombinase and which become the crossover regions during the site-specific recombination event. Examples of sequence-specific recombinase target sites include, but are not limited to, lox sites, frt sites, ATT sites and DIF sites. The sites may be symmetric or asymmetric sites. In preferred embodiments, the sites confer directionality to the recombination reaction. One skilled in the art would appreciate that genes encoding recombinases should be expressed at suitable level, high enough to promote the desired recombination events but not too high so that recombined genetic elements may be stable. Accordingly, recombinase enzymes are preferably under control of an inducible promoter to limit undesired recombination events. In an exemplary embodiment, the host cell is modified to express a set of recombinase enzymes that act on the recombination sites resulting in one or more recombination events. In some embodiments, a host genome may be genetically modified to remove one or more sequences in its genome that are identical or similar to the recombination sites present in the exogenous genetic elements to be recombined. In other embodiments, the host cell is genetically modified to remove one or more restriction sites that are used to promote recombination between different genetic elements within the genetic elements to be recombined. In other embodiments, the host cell may be genetically modified to express a specific restriction enzyme, topoisomerase, repair enzymes and the like. In some embodiments, a plurality of exogenous nucleic acid sequences is introduced into the host cell. Exogenous genetic elements may be introduced sequentially or in combination and may be integrated using multiple rounds of recombination.

In some embodiments, linear synthetic nucleic acid molecules are assembled. Assembled constructs may contain an origin of replication and are capable to replicate in a host cell. In other embodiments, the nucleic acid molecule is inserted in a vector capable of replicating within a host cell or in the natural or synthetic genome of host cell. Nucleic acid molecules may be provided as linear nucleic acid molecules or may be linearized in vivo or excised from larger nucleic acid molecules. In some embodiment, the linear nucleic acid is inserted into a linearized vector. In other embodiments, the linear nucleic acid molecule replaces part of the host cell natural or synthetic genome. In some embodiments, the linear nucleic acid molecule is flanked by a first and a second sequence that are homologous to a first and a second sequence of a linearized vector. Assembly of genetic modules can be achieved by repeated rounds of homologous recombination. This process is repeated until the desired genetic product (such as modified, partially synthetic or fully synthetic genome) has been constructed. In various embodiments, an assembly strategy involves successive rounds of homologous recombination and may involve one or more selectable markers. In a preferred embodiment, the same recombination system is used for each recombination event. In some embodiments, additional genetic elements can be introduced serially into the host cell by transfection techniques such as electroporation. Yet, in other embodiments, genetic elements can be introduced into the host cell by conjugation. In some embodiments, the host cell may be transformed with a vector containing genes encoding the lambda red proteins (gam, bet, exo) under the control of an inducible promoter such as pBAD, plac, Ptrc, Ptet, tsPL promoters to control toxicity of the lambda RED genes.

Selection and isolation of host cells in which the genetic element is expressed may be achieved by any method known in the art. For example, selection may be based directly on the activity of a functional product such as a protein or on the product of metabolic pathway. Selection may also be based on the ability of a host cell to grow in a particular nutritional environment, the production of detectable product, etc. Exemplary selectable markers that may be used in association with the methods described herein include, for example, drug resistance (chloramphenicol, kanamycin, ampicillin, tetracycline, bleomycin, hygromycin, neomycin, zeomycin, gentamycin, streptomycin etc. . . . ), nutritional/auxotrophic (thyA, galK, hisD). In some embodiments, the nucleic acid sequence to be inserted in the host cell comprises a positive or a negative detectable or selectable marker. In some embodiments, the detectable marker is incorporated downstream of the genetic element. In some embodiment each genetic element comprises a detectable marker. For example, a first gene or gene cluster may be linked to a detectable marker (e.g. kanamycine resistance) and a second gene or gene cluster may be linked to a second detectable marker (e.g. ampicillin resistance). In some embodiment, the detectable marker may be excised out of the host cell. The term “detectable marker” refers to a polynucleotide sequence that facilitates the identification of a cell harboring the polynucleotide sequence. In certain embodiments, the detectable marker encodes for a chemiluminescent or fluorescent protein, such as, for example, green fluorescent protein (GFP), enhanced green fluorescent protein (EGFP), Renilla Reniformis green fluorescent protein, GFPmut2, GFPuv4, enhanced yellow fluorescent protein (EYFP), enhanced cyan fluorescent protein (ECFP), enhanced blue fluorescent protein (EBFP), citrine and red fluorescent protein from discosoma (dsRED). In other embodiments, the detectable marker may be an antigenic or affinity tag such as, for example, a polyHis tag, myc, HA, GST, protein A, protein G, calmodulin-binding peptide, thioredoxin, maltose-binding protein, poly arginine, poly His-Asp, FLAG, etc. After recombination is induced, cells expressing the detectable marker are selected. In some embodiments, the selectable marker is an enzyme, a fluorescent marker, a luminescent marker and the like. Examples of suitable detectable markers include, but are not limited to, the green fluorescent protein, the yellow fluorescent protein, the cyan fluorescent protein, luciferase, rhodamine, fluorescein and the like. Accordingly, a host cell should have an appropriate phenotype to allow selection for one or more drug resistance markers encoded on a vector (or to allow detection of one or more detectable markers encoded on a vector). However, any suitable host cell type may be used (e.g., prokaryotic, eukaryotic, bacterial, yeast, insect, mammalian, etc.). For example, host cells may be bacterial cells (e.g., Escherichia coli, Bacillus subtilis, Mycobacterium spp., M. tuberculosis, or other suitable bacterial cells), yeast cells (for example, Saccharomyces spp., Picchia spp., Candida spp., or other suitable yeast species, e.g., S. cerevisiae, C. albicans, S. pombe, etc.), Xenopus cells, mouse cells, monkey cells, human cells, insect cells (e.g., SF9 cells and Drosophila cells), worm cells (e.g., Caenorhabditis spp.), plant cells, or other suitable cells, including for example, transgenic or other recombinant cell lines. In addition, a number of heterologous cell lines may be used, such as Chinese Hamster Ovary cells (CHO). Host cells may be unicellular host cells or multicellular host cells.

In some aspects, a cell line may be modified to remove one or more recombination sites (e.g., by deletion or alteration) from its genome. If one of the recombination site may not be removed because it may affect the viability of the cell, the recombination site may be mutated to decrease the homology so that it will no longer recombine with the recombination site of the genetic elements. If the site is in a coding region, it may be mutated by using alternate codons, and thereby not affecting the protein sequence. Such a modified cell line may therefore host different sets of genetic elements that are configured with the one or more recombination sites that were removed from the host genome. A lack of recombination sites on the host genome reduces the frequency of recombination between the set of genetic elements and the genome, thereby limiting recombination to rearrangements between the genetic elements of interest. In some embodiments, the type of host cell may be determined by the type of vector that is chosen. In some embodiments, the host cell may be chosen depending on the application. A host cell may be modified to have increased activity of one or more ligation and/or recombination functions. In some embodiments, a host cell may be selected on the basis of a high ligation and/or recombination activity. In some embodiments, a host cell may be modified to express (e.g., from the genome or a plasmid expression system) one or more ligase and/or recombinase enzymes.

In some embodiments, the host cell may be engineered to have a modified genome. For example, the host cell may be engineered to have a reduced size genome or a minimal genome. For example, the genome may be smaller by 10%, 20%, 30%, 40%, 50%, 60%, 70% or more. Such an engineered host cell may be adapted to accommodate a plurality of exogenous genetic elements. In some embodiments, the cell has been modified to delete genomic recombination sites. The genomic recombination sites may be reduced by 10-20%, 20-30%, 30-40%, 40-50%, 50-60%, 60-70%, 70-80%, 80-90% or 90-100%. In some embodiments, the genomic recombination sites are reduced by 50% or more. In some embodiments, the genomic recombination sites are reduced by 90% or more.

A host cell may be transformed using any suitable technique (e.g., electroporation, chemical transformation, infection with a viral vector, etc.). Certain host organisms are more readily transformed than others. In some embodiments, all of the nucleic acid fragments and a linearized vector are mixed together and transformed into the host cell in a single step. However, in some embodiments, several transformations may be used to introduce all the fragments and vector into the cell (e.g., several successive transformations using subsets of the fragments). It should be appreciated that the linearized vector is preferably designed to have incompatible ends so that it can only be circularized (and thereby confer resistance to a selectable marker) if the appropriate fragments are cloned into the vector in the designed configuration. This avoids or reduces the occurrence of “empty” vectors after selection. The nucleic acids may be introduced into the host cell by any means known in the art, including, but not limited to, transformation, transfection, electroporation, microinjection, etc. In particular non-limiting embodiments of the invention, one or more nucleic acid may be introduced into a parental host cell, which is then propagated to produce a population of progeny host cells containing the nucleic acids.

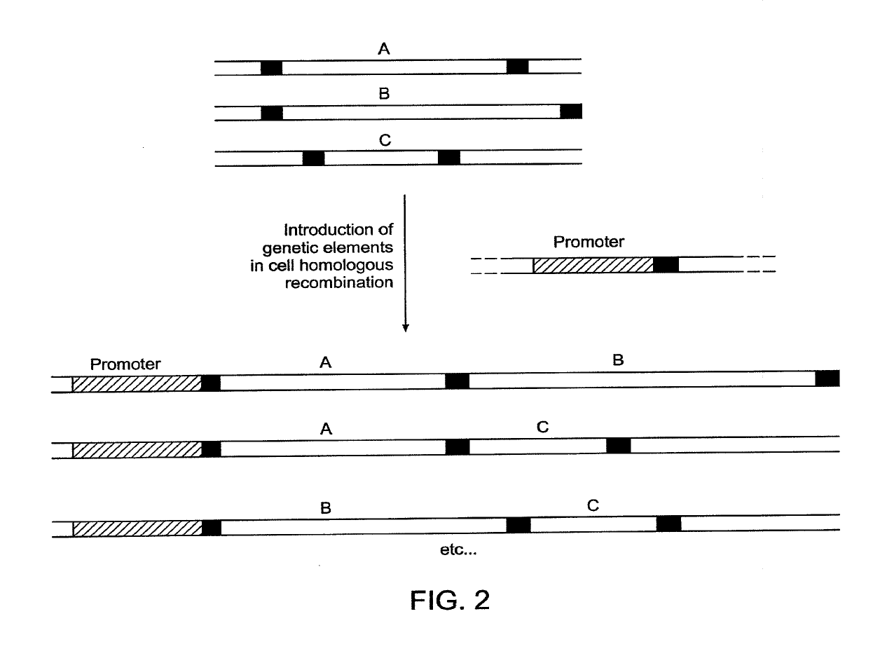

Aspects of the invention provides further a method for the expression of a heterologous nucleotide sequence, wherein the heterologous sequence is introduced into a suitable host cell and the host cell is cultivated under conditions suitable for the expression of the heterologous nucleotide sequence, wherein the expression of the heterologous nucleotide sequence is induced by the restoring the functionality of a promoter by in vivo recombination. Under appropriate recombination conditions, recombination of promoter sequences is promoted in vivo in the host cell due to the recombination sites and a functional promoter is thereby assembled. Host cells hosting the genes that are under the control of the functional promoter sequences can then be exposed to appropriate conditions to induce the transcription of the genes under the control of the functional promoter. Methods of the invention are useful for the preparation and the screening of nucleic sequences libraries. As described above, aspects of the invention allows for the expression of genes of unknown functions. In some aspects, the invention provides methods for generating a cell having a functional diversity by introducing in the cell a plurality of genetic elements associated with multiple recombination sites and allowing or promoting recombination to generate a plurality of predetermined genetic sequences. In some aspects, the invention provides a set of promoter and coding genetic modules associated with recombination sites in an initial configuration. For example, a linear array of promoters and coding sequences flanked with recombination sites is provided in a vector or as a linear nucleic acid fragment. The recombination sites promote rearrangement of promoter and coding modules thereby generating a plurality of novel genetic configurations (FIG. 2). Each genetic configuration may be under the control of a different promoter and can be regulated independently. Appropriate selection and/or screening techniques may be used to identify cells that have a function of interest. The genetic element that is associated with the function of interest may be identified and/or isolated. The genetic element may be amplified, sequenced or cloned. In some embodiments, the genetic element(s) of interest may be integrated into the genome of a host cell. The gene product may be a protein, a metabolite, a RNA, etc. In some embodiments, the genetic element may encode one or more polypeptides. The polypeptide may be expressed, isolated and/or purified by methods known in the art. For example, the polypeptide may be recovered from the growth medium by conventional procedures including, but not limited to, centrifugation, filtration, extraction, spray-drying, evaporation, or precipitation. The polypeptides may be purified by a variety of procedures known in the art including, but not limited to, chromatography (e.g., ion exchange, affinity, hydrophobic, chromatofocusing, and size exclusion), electrophoretic procedures (e.g., preparative isoelectric focusing (IEF), differential solubility (e.g., ammonium sulfate precipitation), or extraction.

Aspects of the invention provide methods for the design and construction of a platform cell. Aspects of the invention provide methods and composition for generating cells having modified functions. More particularly, methods and composition for generating cells having at least one engineered exogenous genetic element and at least one novel function are provided. In some embodiments, the engineered cell comprises a plurality of genetic elements which can be regulated independently. For example, the cell may comprise a plurality of engineered pathways that are under control of different altered promoters. Accordingly, a multifunctional cell may be engineered. In some embodiments, the cell may be customized for a pre-defined function. One should appreciate that such platform cells or chassis cells may be designed for any biotechnological application. Such a modified cell line may be used as a chassis that can host different sets and/or different configurations of genetic elements. In some embodiments, the chassis cell are subjected to a number of rounds of recombination events to create novel functions. Novel functions may include altered activities of existing enzymes, novel regulatory responses (e.g., altered patterns of response to a signal, response to a novel signal, etc., or combinations thereof), novel combinations of enzymes that result in novel pathways (e.g., novel metabolic pathways), other novel functions, or combinations thereof. In some embodiments, selection or screening may be performed on the host cell in which genetic rearrangement occurred. In other embodiments sets of genetic elements are allowed to undergo recombination in chassis cells and are subsequently extracted from the chassis cells. The rearranged set of genetic elements can then be screened in a different system or can be introduced in an alternative cell line, which does not have to be a chassis cell, to be analyzed in vivo. For example, the chassis cell may be E. coli or a recombinant bacterial cell. After recombination of the genetic elements, the rearranged set may be introduced into a different cell line, such as a mammalian cell line (e.g. CHO cells).

Aspects of the invention provide methods by which a cell can be engineered to become a programmable cell. Accordingly, some aspects of the invention relate to a multipurpose cell based microprocessor. In preferred embodiments, the cell based microprocessor comprises a set of biological parts such as genes and/or operons. In some embodiments, the genes and/or operons are nominally all in an off state (defective operons). In preferred embodiments, selected operons are repaired by incorporating predefined correction oligonucleotides into a nucleic acid sequence such as into a plasmid or vector or into the genome. In some embodiments, correction oligonucleotides, having an homology to the target nucleotide sequence (e.g. homology to the regulatory sequence except for the nucleotide(s) to be changed, added or deleted) are incorporated by in vivo or homologous recombination, thereby restoring the activity of the regulatory region. Yet in other embodiments, the correction oligonucleotides having an homology to coding sequences (except for the nucleotide(s) to be changed, added or deleted, are incorporated within the cell nucleic acid sequence by in vivo or homologous recombination.

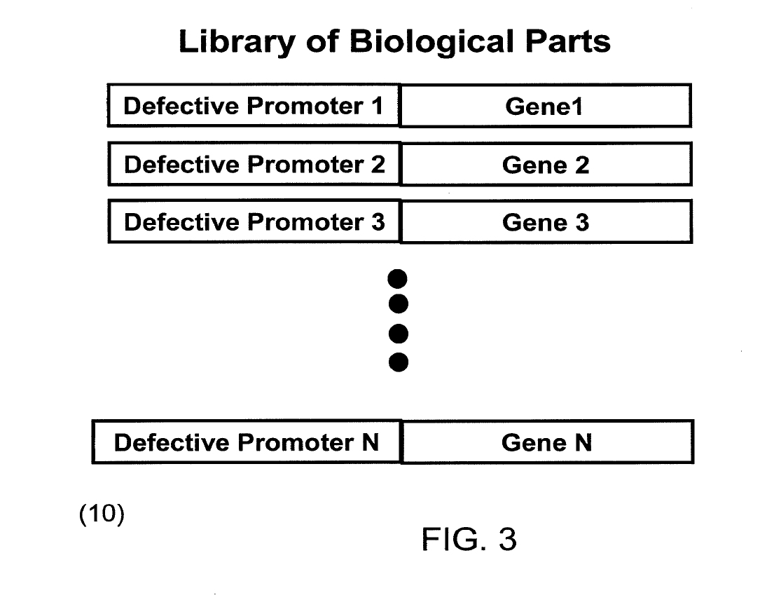

FIG. 3 schematically depicts a library of biological parts (10) such as that kept, for example, by the Registry of Standard Biological Parts but in which the associated promoters or operons which control the genes of said parts have been mutated, typically by a single or a small number of bases, in order to render then non-operable and to switch off the expression of the associated or operably linked gene.

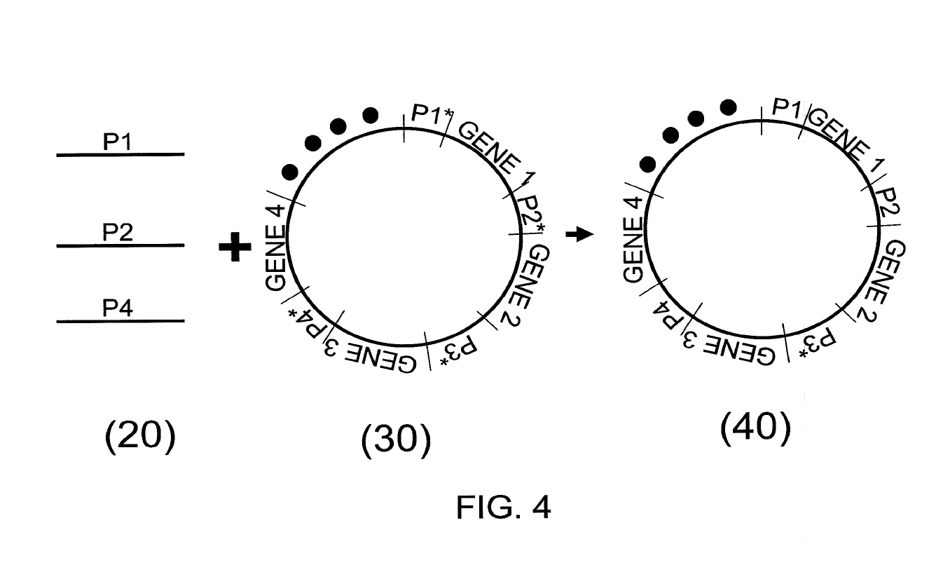

As illustrated in FIG. 4, the microprocessor cell contains in its genome or on a separate plasmid (30) a plurality of biological parts from a nucleic acid library (10). In an exemplary embodiment, correction oligonucleotides (20) can be inserted into the genome or the nucleic acid sequences of the cell by homologous recombination or lambda red mediated recombination. After the correction oligonucleotides (20) are recombined with the genome or plasmid, the oligonucleotide will correct the associated mutated and inoperable promoters thereby rendering them operable. Referring to FIG. 2, each gene on the plasmid (30) is operably linked to a different inoperable or altered promoter p1*, p2*, p3* etc. In some embodiments, the altered promoter P* have been mutated such that the promoters are inoperable (e.g. do not function to recruit polymerase). Accordingly, as illustrated in FIG. 2 none of the genes (represented by gene 1, gene 2, gene 3 etc.) are expressed. In some embodiments, a genetic circuit is assembled from a selected number of available part. For example, if genes 1, 2 and 4 represent three genes needed in the genetic circuit, one would appreciate that by inserting correction oligonucleotides p1, p2 and p4 (20) into plasmid (30), a plasmid (40) in which promoters p1, p2, and p4 are now operably linked to gene 1, gene 2 and gene 4, respectively, while promoter p*3 on the plasmid remains inoperable. One skilled in the art would appreciate that this way a genetic circuit containing operable gene 1, gene 2 and gene 3 is assembled, allowing the cell to perform a desired function. However, one could program plasmid (30) to perform a wholly different function and constitute a wholly different genetic circuit based on other genes on the plasmid by introducing a different set of programming oligonucleotides.

Aspects of the invention may be used for industrial applications, pharmaceutical applications, agricultural applications, environmental applications, etc. For example, genes of interest may encode therapeutic proteins or peptides (e.g growth factors, hormones, cytokines, ligands, receptors and inhibitors, antibodies or vaccines). Genes may encode enzymes or other commercially important proteins or peptides.

Aspects of the invention may be used to synthesize and regulate the expression of one or more exogenous gene product. In some embodiments, gene products may be polypeptides, preferably enzymes of an engineered metabolic pathway. A metabolic pathway is a series of chemical reactions, such as catabolic reaction or anabolic reactions that are catalyzed by a number of enzymes. Most metabolic pathways comprise a rate-limiting enzymatic step which regulate the pathway. One should therefore appreciate that methods and compositions described herein allow for the expression of optimal expression levels of each metabolic enzyme for the production of the metabolites of interest in a host cell. The expression levels of each enzyme may be modulated by the library of promoters or by the library of disrupted promoters. Accordingly, aspects of the invention may be used to synthesize and regulate levels of one or more metabolites (e.g. intermediates or products) for agricultural, industrial, pharmaceutical, or other purposes.

### EQUIVALENTS

While specific embodiments of the subject invention have been discussed, the above specification is illustrative and not restrictive. Many variations of the invention will become apparent to those skilled in the art upon review of this specification. The full scope of the invention should be determined by reference to the claims, along with their full scope of equivalents, and the specification, along with such variations.

### INCORPORATIONS BY REFERENCE

All publications, patents and patent applications mentioned herein are hereby incorporated by reference in their entirety as if each individual publication or patent was specifically and individually indicated to be incorporated by reference. In case of conflict, the present application, including any definitions herein, will control.

1. A method for expressing at least one polypeptide of interest in a host cell, the method comprising:
a. introducing a set of genetic elements in a host cell, the set of genetic elements comprising at least one regulatory sequence and at least one coding nucleic acid sequence of interest, wherein the set of genetic elements comprises recombination sites therebetween;
b. exposing the cell under conditions promoting recombination;
c. rearranging the set of genetic elements by allowing recombination between recombination sites; and
d. selecting the host cell having expressing the at least one polypeptide of interest.

2. The method of claim 1 wherein the regulatory sequence is a promoter sequence.

3. The method of claim 1 wherein the genetic elements are on the same nucleic acid or on different nucleic acids.

4. (canceled)

5. The method of claim 1 wherein the genetic elements are on a plasmid, a vector or are integrated in the genome of the host cell.

6. (canceled)

7. The method of claim 1 wherein the expression of the at least one polypeptide of interest is modulated by rearranging the regulatory sequence.

8. The method of claim 1 wherein at least one regulatory sequence is disrupted.

9. The method of claim 1 wherein expression of selected coding sequences is modulated by restoring the activity of the at least one regulatory sequence.

10. The method of claim 9 wherein the disrupted regulatory sequence comprises a 3′ segment having a recombination site at its 5′ end and a 5′ segment having a recombination site at its 3′ end and wherein the activity of the regulatory sequence is restored by recombination.

11. (canceled)

12. The method of claim 1 wherein the at least one regulatory sequence is a library of promoters.

13. (canceled)

14. The method of claim 12 wherein the library of promoters is a library of promoter variants.

15. The method of claim 1 wherein the at least one coding sequence is a library of unrelated coding sequences.

16-22. (canceled)

23. A method for manipulating the genome of host cell to produce at least one exogenous gene product, the method comprising:
a. providing a host cell capable of performing site directed recombination;
b. providing two or more genetic elements, wherein a first genetic element comprises a genetically disrupted promoter sequence that is operably linked to a second genetic element comprising at least part of a coding sequence;
c. contacting the host cell with the genetic elements;
d. restoring the activity of the disrupted promoter sequence by site directed recombination;
e. selecting a host cell in which the recombination has occurred; and
f. producing the at least one exogenous gene product.

24. The method of claim 23 wherein the gene product is not expressed from said genetic element when the promoter is disrupted.

25. The method of claim 24 wherein the promoter is disrupted by a sequence alteration.

26-48. (canceled)

49. The method of claim 23 wherein the second genetic element comprises a cluster of genes.

50. The method of claim 49 wherein the cluster of gene codes for metabolic enzymes from a metabolic pathway.

51-86. (canceled)

87. A method of producing a programmable engineered cell capable of expressing at least one nucleic acid sequence, the method comprising:
a. providing a cell comprising at least one exogenous nucleic acid sequence wherein the at least one nucleic acid sequence is linked to a regulatory nucleic acid sequence;
b. providing at least one predefined oligonucleotide sequence homologous to the at least part of the regulatory nucleic acid sequence;
c. exposing the cell to conditions promoting oligonucleotide-directed recombination; and
d. selecting a cell expressing the at least one nucleic acid sequence.

88. The method of claim 87 wherein the at least one nucleic acid sequence is operably linked to the regulatory sequence.

89. The method of claim 88 wherein the regulatory sequence is a promoter sequence and the regulatory sequence is disrupted.

90-91. (canceled)

92. The method of claim 87 wherein the at least one nucleic acid sequence is a library of genes and the at least one oligonucleotide is a library of predefined oligonucleotides sequences.

93. The method of claim 87 wherein at least part of the regulatory sequence is replaced by homologous recombination.

94. The method of claim 93 wherein recombination between homologous sequences occurs in parallel or serially.

95-97. (canceled)
